(19) United States
(12) Patent Application Publication
(10) Pub. No.: US 2015/0257653 A1
(43) Pub. Date: Sep. 17, 2015

(54) DEVICE, SYSTEM, AND METHOD FOR DETERMINING BLOOD PRESSURE IN A MAMMALIAN SUBJECT

(71) Applicant: Elwha LLC, Bellevue, WA (US)

(72) Inventors: Roderick A. Hyde, Redmond, WA (US); Lowell L. Wood, JR., Bellevue, WA (US)

(21) Appl. No.: 14/211,811

(22) Filed: Mar. 14, 2014

(51) Int. Cl. A61B 5/021; A61B 5/024

(52) U.S. Cl. CPC A61B 5/021; A61B 5/02433

(57) ABSTRACT

Devices, systems, and methods are disclosed herein for remotely determining blood pressure in a mammalian subject. A method for remotely determining blood pressure in a mammalian subject is disclosed that includes emitting multiple radiation pulses to one or more locations on a skin surface of the mammalian subject; and determining the blood pressure in the subject based on a calculation of timing of one or more heartbeats relative to timing of a first blood pressure pulse and timing of a second blood pressure pulse.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

Devices, systems, and methods are disclosed herein for remotely determining blood pressure in a mammalian subject. A method for remotely determining blood pressure in a mammalian subject is disclosed that includes emitting multiple micropower impulse radar (MIR) pulses from one or more radiation pulse generator transmitters to a first location on a skin surface of the mammalian subject; utilizing one or more contact-free tissue probes including one or more MIR pulse detectors responsive to scattered multiple MIR pulses in an environment of the mammalian subject; determining one or more heartbeats of the mammalian subject by detecting multiple MIR pulses scattered to the one or more MIR pulse detectors; detecting at one or more optical radiation detectors a first reflected optical radiation from blood at a second location at a skin surface of the subject and a second reflected optical radiation from blood at a third location at the skin surface of the subject; determining the timing of a first blood pressure pulse at the second location by comparing levels of the first reflected optical radiation measured at different times; determining the timing of a second blood pressure pulse at the third location by comparing levels of the second reflected optical radiation measured at different times; and determining the blood pressure in the subject based on a calculation of timing of the one or more heartbeats relative to timing of the first blood pressure pulse and the timing of the second blood pressure pulse. In some aspects, the method may further include utilizing the one or more radiation pulse generator transmitters and the one or more tissue probes mounted to an exterior surface in an environment of the subject.

In some aspects, an origin of the first blood pressure pulse and the second blood pressure pulse are a single origin. The method may include directing a first optical light source toward the second location for generation of the first reflected optical radiation. The method may include directing a second optical light source toward the third location for generation of the second reflected optical radiation. In some aspects, the first and second reflected optical radiation is infrared radiation. The first and second reflected optical radiation may be two or more wavelengths of infrared radiation. The optical radiation includes, but is not limited to, ambient room radiation, ambient solar radiation, artificial room radiation, an optical light source, LED radiation, incandescent radiation, or fluorescent radiation. The optical light source may include an incoherent optical source. The method may include detecting the first and second reflected optical radiation from the second location and the third location at two or more time points. The method may include detecting the first and second reflected optical radiation from the second location and the third location with a high frame rate camera. The method may further include enhancing video resolution from the high frame rate camera. The method may include measuring systole and diastole at the heart of the subject with the scattered multiple MIR pulses to the one or more MIR pulse detectors. The method may include identifying the second location and the third location from a video image on a camera. The second location and the third location may include predetermined target locations. The method may further include identifying the second location and the third location relative to the first location of the heart of the subject, and applying a corrected calculation to determine the blood pressure in the subject. The method may further include utilizing facial recognition from the camera to identify the subject. The method may include utilizing the tissue probe and the one or more optical radiation detectors mounted to an exterior surface in the environment of the mammalian subject. The exterior surface may include, but is not limited to, a wall, ceiling, furniture, or computer in the environment.

A method for remotely determining blood pressure in a subject includes emitting multiple micropower impulse radar (MIR) pulses from one or more radiation pulse generator transmitters to a first location on a skin surface of the mammalian subject; utilizing one or more contact-free tissue probes including one or more MIR pulse detectors responsive to scattered multiple MIR pulses in an environment of the mammalian subject; determining one or more heartbeats of the mammalian subject by detecting multiple MIR pulses scattered to the one or more MIR pulse detectors; utilizing the one or more radiation pulse generator transmitters and the one or more tissue probes mounted to an exterior surface in an environment of the subject; detecting at one or more optical radiation detectors a first reflected optical radiation from blood at a second location at a skin surface of the subject and a second reflected optical radiation from blood at a third location at the skin surface of the subject; determining the timing of a first blood pressure pulse at the second location by comparing levels of the first reflected optical radiation measured at different times; determining the timing of a second blood pressure pulse at the third location by comparing levels of the second reflected optical radiation measured at different times; determining the blood pressure in the subject based on a calculation of timing of the one or more heartbeats relative to timing of the first blood pressure pulse and timing of the second blood pressure pulse.

A device is provided for use in the method for remotely determining blood pressure in a mammalian subject is disclosed. A device includes one or more radiation pulse generator transmitters emitting multiple micropower impulse radar (MIR) pulses to a first location on a skin surface of the mammalian subject; one or more contact-free tissue probes including one or more MIR pulse detectors responsive to scattered multiple MIR pulses in an environment of the mammalian subject, wherein the scattered multiple MIR pulses to the one or more MIR pulse detectors are operable to measure one or more heart beats of the mammalian subject; one or more optical radiation detectors operable to measure a first reflected optical radiation from blood at a second location at a skin surface of the subject and a second reflected optical radiation from blood at a third location at the skin surface of the subject; a controller operable to determine the timing of a first blood pressure pulse at the second location by comparing levels of the first reflected optical radiation measured at different times and operable to determine the timing of a second blood pressure pulse at the third location by comparing levels of the second reflected optical radiation measured at different times; and operable to determine the blood pressure in the subject based on a calculation of timing of the one or more heartbeats relative to timing of the first blood pressure pulse and the timing of the second blood pressure pulse.

A device is provided for use in the method for remotely determining blood pressure in a mammalian subject is disclosed. A device includes one or more radiation pulse generator transmitters emitting multiple micropower impulse radar (MIR) pulses to a first location on a skin surface of the mammalian subject; one or more contact-free tissue probes including one or more MIR pulse detectors responsive to scattered multiple MIR pulses in an environment of the mammalian subject, wherein the scattered multiple MIR pulses to the one or more MIR pulse detectors are operable to measure one or more heart beats of the mammalian subject; one or more optical radiation detectors operable to measure a first reflected optical radiation from blood at a second location at a skin surface of the subject and a second reflected optical radiation from blood at a third location at the skin surface of the subject; a controller operable to determine the timing of a first blood pressure pulse at the second location by comparing levels of the first reflected optical radiation measured at different times and operable to determine the timing of a second blood pressure pulse at the third location by comparing levels of the second reflected optical radiation measured at different times; and operable to determine the blood pressure in the subject based on a calculation of timing of the one or more heartbeats relative to timing of the first blood pressure pulse and the timing of the second blood pressure pulse.

In some aspects, an origin of the first blood pressure pulse and the second blood pressure pulse is a single origin. In some aspects, the first and second reflected optical radiation is infrared radiation. The first and second reflected optical radiation may include two or more wavelengths of infrared radiation. The optical radiation may include, but is not limited to, ambient room radiation, ambient solar radiation, artificial room radiation, an optical light source, LED radiation, incandescent radiation, or fluorescent radiation. The optical light source may include an incoherent optical source. The second location and the third location may include predetermined target locations identified from a video image on a camera. The tissue probe and the one or more optical radiation detectors may be mounted to an exterior surface in the environment of the mammalian subject.

A system is provided that includes a device including: one or more radiation pulse generator transmitters emitting multiple micropower impulse radar (MIR) pulses to a first location on a skin surface of the mammalian subject; one or more contact-free tissue probes including one or more MIR pulse detectors responsive to scattered multiple MIR pulses in an environment of the mammalian subject, wherein the scattered multiple MIR pulses to the one or more MIR pulse detectors are operable to measure one or more heart beats of the mammalian subject; one or more optical radiation detectors operable to measure a first reflected optical radiation from blood at a second location at a skin surface of the subject and a second reflected optical radiation from blood at a third location at the skin surface of the subject; a controller operable to determine the timing of a first blood pressure pulse at the second location by comparing levels of the first reflected optical radiation measured at different times and operable to determine the timing of a second blood pressure pulse at the third location by comparing levels of the second reflected optical radiation measured at different times; and operable to determine the blood pressure in the subject based on a calculation of timing of the one or more heartbeats relative to timing of the first blood pressure pulse and the timing of the second blood pressure pulse, wherein the one or more radiation pulse generator transmitters and the one or more tissue probes are operable to be mounted to an exterior surface in an environment of the subject.

A method for remotely determining blood pressure in a mammalian subject is provided that includes emitting multiple micropower impulse radar (MIR) pulses from one or more radiation pulse generator transmitters to a first location on a skin surface of the mammalian subject; utilizing one or more contact-free tissue probes including one or more MIR pulse detectors responsive to scattered multiple MIR pulses in an environment of the mammalian subject; determining one or more heartbeats of the mammalian subject by detecting multiple MIR pulses scattered to the one or more MIR pulse detectors; detecting at one or more optical radiation detectors a first reflected optical radiation from blood at a second location at a skin surface of the subject; determining the timing of a first blood pressure pulse at the second location by comparing levels of the first reflected optical radiation measured at different times; determining the blood pressure in the subject based on a calculation of the timing of the one or more heartbeats relative to the timing of the first blood pressure pulse. The method may include directing a first optical light source toward the second location for generation of the first reflected optical radiation. In some aspects, the first reflected optical radiation is infrared radiation. The optical radiation includes, but is not limited to, ambient room radiation, ambient solar radiation, artificial room radiation, radiation from an optical light source, LED radiation, incandescent radiation, or fluorescent radiation. In some aspects, the optical light source may include an incoherent optical source. The method may include detecting the MIR pulse from the first location and the first reflected optical radiation from the second location at two or more time points. The method may include detecting the first reflected optical radiation from the second location with a high frame rate camera. The method may include enhancing video resolution from the high frame rate camera. The method may include measuring systole and diastole at the heart of the subject with the scattered multiple MIR pulses to the one or more MIR pulse detectors. The method may include identifying the second location from a video image on a camera. The second location may include a predetermined target location. The method may include identifying the second location relative to the first location of the heart of the subject, and applying a corrected calculation to determine the blood pressure in the subject. The method may include utilizing facial recognition from the camera to identify the subject. The method may include utilizing the tissue probe and the one or more optical radiation detectors mounted to an exterior surface in the environment of the mammalian subject. The exterior surface may include, but is not limited to, a wall, ceiling, furniture, or computer in the environment.

A method for remotely determining blood pressure in a mammalian subject is provided that includes emitting multiple micropower impulse radar (MIR) pulses from one or more radiation pulse generator transmitters to a first location on a skin surface of the mammalian subject; utilizing one or more contact-free tissue probes including one or more MIR pulse detectors responsive to scattered multiple MIR pulses in an environment of the mammalian subject; determining one or more heartbeats of the mammalian subject by detecting multiple MIR pulses scattered to the one or more MIR pulse detectors; detecting at one or more optical radiation detectors a first reflected optical radiation from blood at a second location at a skin surface of the subject; determining the timing of a first blood pressure pulse at the second location by comparing levels of the first reflected optical radiation measured at different times; determining the blood pressure in the subject based on a calculation of the timing of the one or more heartbeats relative to the timing of the first blood pressure pulse; and utilizing the one or more radiation pulse generator transmitters and the one or more tissue probes mounted to an exterior surface in an environment of the subject.

A device is provided that includes one or more radiation pulse generator transmitters emitting multiple micropower impulse radar (MIR) pulses to a first location on a skin surface of the mammalian subject; one or more contact-free tissue probes including one or more MIR pulse detectors responsive to scattered multiple MIR pulses in an environment of the mammalian subject, wherein the scattered multiple MIR pulses to the one or more MIR pulse detectors are operable to measure one or more heart beats of the mammalian subject; one or more optical radiation detectors operable to measure a first reflected optical radiation from blood at a second location at a skin surface of the subject; a controller operable to determine the timing of a first blood pressure pulse at the second location by comparing levels of the first reflected optical radiation measured at different times; and scattered multiple MIR operable to determine the blood pressure in the subject based on a calculation of the timing of the one or more heartbeats relative to the timing of the first blood pressure pulse. The first reflected optical radiation may be infrared radiation. The first reflected optical radiation may be two or more wavelengths of infrared radiation. The optical radiation includes, but is not limited to, ambient room radiation, ambient solar radiation, artificial room radiation, radiation from an optical light source, LED radiation, incandescent radiation, or fluorescent radiation. The optical light source may be an incoherent optical source. The second location includes a predetermined target location identified from a video image on a camera. The tissue probe and the one or more optical radiation detectors may be mounted to an exterior surface in the environment of the mammalian subject.

A system is provided that includes: a device including: one or more radiation pulse generator transmitters emitting multiple micropower impulse radar (MIR) pulses to a first location on a skin surface of the mammalian subject; one or more contact-free tissue probes including one or more MIR pulse detectors responsive to scattered multiple MIR pulses in an environment of the mammalian subject, wherein the scattered multiple MIR pulses to the one or more MIR pulse detectors are operable to measure one or more heart beats of the mammalian subject; one or more optical radiation detectors operable to measure a first reflected optical radiation from blood at a second location at a skin surface of the subject; a controller operable to determine the timing of a first blood pressure pulse at the second location by comparing levels of the first reflected optical radiation measured at different times; and operable to determine the blood pressure in the subject based on a calculation of the timing of the one or more heartbeats relative to the timing of the first blood pressure pulse, wherein the one or more radiation pulse generator transmitters and the one or more tissue probes are operable to be mounted to an exterior surface in an environment of the subject.

A method for remotely determining blood pressure in a mammalian subject is provided that includes utilizing one or more contact-free tissue probes including one or more optical radiation detectors responsive to reflected optical radiation in an environment of the mammalian subject; detecting at the one or more optical radiation detectors a first reflected optical radiation from blood at a first location at a skin surface of the subject and a second reflected optical radiation from blood at a second location at the skin surface of the subject; determining the timing of a first blood pressure pulse at the first location by comparing levels of the first reflected optical radiation measured at different times; determining the timing of a second blood pressure pulse at the second location by comparing levels of the second reflected optical radiation measured at different times; and determining the blood pressure in the subject based on a calculation of the timing of the first blood pressure pulse relative to the timing of the second blood pressure pulse. In some aspects, an origin of the first blood pressure pulse and the second blood pressure pulse are a single origin. The method may include directing a first optical light source toward the first location for generation of the first reflected optical radiation. The method may include directing a second optical light source toward the second location for generation of the second reflected optical radiation. The first and second reflected optical radiation may include infrared radiation. The first and second reflected optical radiation may include two or more wavelengths of infrared radiation. The optical radiation includes, but is not limited to, ambient room radiation, ambient solar radiation, artificial room radiation, radiation from an optical light source, LED radiation, incandescent radiation, or fluorescent radiation. The optical light source may include an incoherent optical source. The method may include detecting the first and second reflected optical radiation from the first location and the second location at two or more time points. The method may include detecting the first and second reflected optical radiation from the first location and the second location with a high frame rate camera. The method may include enhancing video resolution from the high frame rate camera. The method may include identifying the first location and the second location from a video image on a camera. The first location and the second location may include predetermined target locations. The method may include utilizing facial recognition from the camera to identify the subject. The method may include utilizing the tissue probe and the one or more optical radiation detectors mounted to an exterior surface in the environment of the mammalian subject. The exterior surface may include, but is not limited to, a wall, ceiling, furniture, or computer in the environment.

A method for remotely determining blood pressure in a mammalian subject is provided that includes: utilizing one or more contact-free tissue probes including one or more optical radiation detectors responsive to reflected optical radiation in an environment of the mammalian subject; utilizing the one or more contact-free tissue probes including the one or more optical radiation detectors mounted to an exterior surface in an environment of the subject; detecting at the one or more optical radiation detectors a first reflected optical radiation from blood at a first location at a skin surface of the subject and a second reflected optical radiation from blood at a second location at the skin surface of the subject; determining the timing of a first blood pressure pulse at the first location by comparing levels of the first reflected optical radiation measured at different times; determining the timing of a second blood pressure pulse at the second location by comparing levels of the second reflected optical radiation measured at different times; and determining the blood pressure in the subject based on a calculation of the timing of the first blood pressure pulse relative to the timing of the second blood pressure pulse.

A device is provided that includes: one or more optical radiation detectors operable to measure a first reflected optical radiation from blood at a first location at a skin surface of the subject and a second reflected optical radiation from blood at a second location at the skin surface of the subject; a controller operable to determine the timing of a first blood pressure pulse at the first location by comparing levels of the first reflected optical radiation measured at different times, and operable to determine the timing of a second blood pressure pulse at the second location by comparing levels of the second reflected optical radiation measured at different times, and operable to determine the blood pressure in the subject based on a calculation of the timing of the first blood pressure pulse relative to the timing of the second blood pressure pulse. In some aspects, the origin of the first blood pressure pulse and the second blood pressure pulse may include a single origin. The first and second reflected optical radiation may be infrared radiation. The first and second reflected optical radiation may be two or more wavelengths of infrared radiation. The optical radiation may include, but is not limited to, ambient room radiation, ambient solar radiation, artificial room radiation, radiation from an optical light source, LED radiation, incandescent radiation, or fluorescent radiation. The optical light source may include an incoherent optical source. In some aspects, the first location and the second location may include predetermined target locations identified from a video image on a camera. In some aspects, the tissue probe and the one or more optical radiation detectors may be mounted to an exterior surface in the environment of the mammalian subject.

A system is provided that includes: a device including one or more optical radiation detectors operable to measure a first reflected optical radiation from blood at a first location at a skin surface of the subject and a second reflected optical radiation from blood at a second location at the skin surface of the subject; a controller operable to determine the timing of a first blood pressure pulse at the first location by comparing levels of the first reflected optical radiation measured at different times, and operable to determine the timing of a second blood pressure pulse at the second location by comparing levels of the second reflected optical radiation measured at different times, and operable to determine the blood pressure in the subject based on a calculation of the timing of the first blood pressure pulse relative to the timing of the second blood pressure pulse, wherein the one or more radiation pulse generator transmitters and the one or more tissue probes are operable to be mounted to an exterior surface in an environment of the subject.

A method for remotely determining blood pressure in a mammalian subject is provided that includes: utilizing one or more tissue probes including one or more pulse detectors responsive to reflected multiple pulses in an environment of the mammalian subject, wherein the reflected multiple pulses to the one or more pulse detectors are operable to measure one or more heart beats of the mammalian subject; utilizing one or more contact-free tissue probes including one or more optical radiation detectors responsive to reflected optical radiation in an environment of the mammalian subject; detecting at the one or more optical radiation detectors a first reflected optical radiation from blood at a first location at a skin surface of the subject and a second reflected optical radiation from blood at a second location at the skin surface of the subject; determining the timing of a first blood pressure pulse at the first location by comparing levels of the first reflected optical radiation measured at different times; determining the timing of a second blood pressure pulse at the second location by comparing levels of the second reflected optical radiation measured at different times; and determining the blood pressure in the subject based on a calculation of timing of the one or more heartbeats relative to the timing of the first blood pressure pulse relative to the timing of the second blood pressure pulse. In some aspects, an origin of the first blood pressure pulse and the second blood pressure pulse may include a single origin.

The method may include directing a first optical light source toward the first location for generation of the first reflected optical radiation. The method may include directing a second optical light source toward the second location for generation of the second reflected optical radiation. The first and second reflected optical radiation may include infrared radiation. The first and second reflected optical radiation may include two or more wavelengths of infrared radiation. The optical radiation may include, but is not limited to, ambient room radiation, ambient solar radiation, artificial room radiation, radiation from an optical light source, LED radiation, incandescent radiation, or fluorescent radiation. The optical light source may include an incoherent optical source. The method may include detecting the first and second reflected optical radiation from the first location and the second location at two or more time points. The method may include detecting the first and second reflected optical radiation from the first location and the second location with a high frame rate camera. The method may further include enhancing video resolution from the high frame rate camera. The method may include measuring systole and diastole at the heart of the subject with the reflected multiple pulses to the one or more pulse detectors. The method may include identifying the first location and the second location from a video image on a camera. The first location and the second location may include predetermined target locations. The method may include identifying the first location and the second location relative to a location of the heart of the subject, and applying a corrected calculation to determine the blood pressure in the subject. The method may include utilizing facial recognition from the camera to identify the subject. The method may include utilizing the tissue probe and the one or more optical radiation detectors mounted to an exterior surface in the environment of the mammalian subject. The exterior surface may include, but is not limited to, a wall, ceiling, furniture, or computer in the environment.

A method for remotely determining blood pressure in a mammalian subject is provided that includes: utilizing one or more tissue probes including one or more pulse detectors responsive to reflected multiple pulses in an environment of the mammalian subject, wherein the reflected multiple pulses to the one or more pulse detectors are operable to measure one or more heart beats of the mammalian subject; utilizing one or more contact-free tissue probes including one or more optical radiation detectors responsive to reflected optical radiation in an environment of the mammalian subject; utilizing the one or more contact-free tissue probes including the one or more optical radiation detectors mounted to an exterior surface in an environment of the subject; detecting at the one or more optical radiation detectors a first reflected optical radiation from blood at a first location at a skin surface of the subject and a second reflected optical radiation from blood at a second location at the skin surface of the subject; determining the timing of a first blood pressure pulse at the second location by comparing levels of the first reflected optical radiation measured at different times; determining the timing of a second blood pressure pulse at the third location by comparing levels of the second reflected optical radiation measured at different times; and determining the blood pressure in the subject based on a calculation of timing of the one or more heartbeats relative to the timing of the first blood pressure pulse and the timing of the second blood pressure pulse.

A device is provided that includes: one or more radiation pulse generator transmitters emitting multiple power pulses to a first location on a skin surface of the mammalian subject; one or more tissue probes including one or more power pulse detectors responsive to reflected power pulses in an environment of the mammalian subject, wherein the reflected multiple power pulses to the one or more power pulse detectors are operable to measure one or more heart beats of the mammalian subject; one or more optical radiation detectors operable to measure a first reflected optical radiation from blood at a second location at a skin surface of the subject and a second reflected optical radiation from blood at a third location at the skin surface of the subject; a controller operable to determine the timing of a first blood pressure pulse at the second location by comparing levels of the first reflected optical radiation measured at different times, and operable to determine the timing of a second blood pressure pulse at the third location by comparing levels of the second reflected optical radiation measured at different times, and operable to determine the blood pressure in the subject based on a calculation of timing of the one or more heartbeats relative to the timing of the first blood pressure pulse and the timing of the second blood pressure pulse. In some aspects, an origin of the first blood pressure pulse and the second blood pressure pulse may include a single origin. The first and second reflected optical radiation may include infrared radiation. The first and second reflected optical radiation may include two or more wavelengths of infrared radiation. The optical radiation may include, but is not limited to, ambient room radiation, ambient solar radiation, artificial room radiation, radiation from an optical light source, LED radiation, incandescent radiation, or fluorescent radiation. The optical light source may include an incoherent optical source. The second location and the third location may include predetermined target locations identified from a video image on a camera. In some aspects, the tissue probe and the one or more optical radiation detectors may be mounted to an exterior surface in the environment of the mammalian subject.

A system is provided that includes: a device including one or more radiation pulse generator transmitters emitting multiple power pulses to a first location on a skin surface of the mammalian subject; one or more tissue probes including one or more power pulse detectors responsive to reflected power pulses in an environment of the mammalian subject, wherein the reflected multiple power pulses to the one or more power pulse detectors are operable to measure one or more heart beats of the mammalian subject; one or more optical radiation detectors operable to measure a first reflected optical radiation from blood at a second location at a skin surface of the subject and a second reflected optical radiation from blood at a third location at the skin surface of the subject; a controller operable to determine the timing of a first blood pressure pulse at the second location by comparing levels of the first reflected optical radiation measured at different times, and operable to determine the timing of a second blood pressure pulse at the third location by comparing levels of the second reflected optical radiation measured at different times, and operable to determine the blood pressure in the subject based on a calculation of timing of the one or more heartbeats relative to the timing of the first blood pressure pulse and the timing of the second blood pressure pulse, wherein the one or more radiation pulse generator transmitters and the one or more tissue probes are operable to be mounted to an exterior surface in an environment of the subject.

An article of manufacture is provided that includes: one or more non-transitory machine-readable data storage media bearing one or more instructions for: emitting multiple micropower impulse radar (MIR) pulses from one or more radiation pulse generator transmitters to a first location on a skin surface of the mammalian subject; utilizing one or more contact-free tissue probes including one or more MIR pulse detectors responsive to scattered multiple MIR pulses in an environment of the mammalian subject; determining one or more heartbeats of the mammalian subject by detecting multiple MIR pulses scattered to the one or more MIR pulse detectors; detecting at one or more optical radiation detectors a first reflected optical radiation from blood at a second location at a skin surface of the subject and a second reflected optical radiation from blood at a third location at the skin surface of the subject; determining the timing of a first blood pressure pulse at the second location by comparing levels of the first reflected optical radiation measured at different times; determining the timing of a second blood pressure pulse at the third location by comparing levels of the second reflected optical radiation measured at different times; and determining the blood pressure in the subject based on a calculation of timing of the one or more heartbeats relative to timing of the first blood pressure pulse and the timing of the second blood pressure pulse.

An article of manufacture is provided that includes: one or more non-transitory machine-readable data storage media bearing one or more instructions for: emitting multiple micropower impulse radar (MIR) pulses from one or more radiation pulse generator transmitters to a first location on a skin surface of the mammalian subject; utilizing one or more contact-free tissue probes including one or more MIR pulse detectors responsive to scattered multiple MIR pulses in an environment of the mammalian subject; determining one or more heartbeats of the mammalian subject by detecting multiple MIR pulses scattered to the one or more MIR pulse detectors; detecting at one or more optical radiation detectors a first reflected optical radiation from blood at a second location at a skin surface of the subject; determining the timing of a first blood pressure pulse at the second location by comparing levels of the first reflected optical radiation measured at different times; determining the blood pressure in the subject based on a calculation of the timing of the one or more heartbeats relative to the timing of the first blood pressure pulse.

An article of manufacture is provided that includes: one or more non-transitory machine-readable data storage media bearing one or more instructions for: utilizing one or more contact-free tissue probes including one or more optical radiation detectors responsive to reflected optical radiation in an environment of the mammalian subject; detecting at the one or more optical radiation detectors a first reflected optical radiation from blood at a first location at a skin surface of the subject and a second reflected optical radiation from blood at a second location at the skin surface of the subject; determining the timing of a first blood pressure pulse at the first location by comparing levels of the first reflected optical radiation measured at different times; determining the timing of a second blood pressure pulse at the second location by comparing levels of the second reflected optical radiation measured at different times; and determining the blood pressure in the subject based on a calculation of the timing of the first blood pressure pulse relative to the timing of the second blood pressure pulse.

An article of manufacture is provided that includes: one or more non-transitory machine-readable data storage media bearing one or more instructions for: utilizing one or more tissue probes including one or more pulse detectors responsive to reflected multiple pulses in an environment of the mammalian subject, wherein the reflected multiple pulses to the one or more pulse detectors are operable to measure one or more heart beats of the mammalian subject; utilizing one or more contact-free tissue probes including one or more optical radiation detectors responsive to reflected optical radiation in an environment of the mammalian subject; detecting at the one or more optical radiation detectors a first reflected optical radiation from blood at a first location at a skin surface of the subject and a second reflected optical radiation from blood at a second location at the skin surface of the subject; determining the timing of a first blood pressure pulse at the first location by comparing levels of the first reflected optical radiation measured at different times; determining the timing of a second blood pressure pulse at the second location by comparing levels of the second reflected optical radiation measured at different times; and determining the blood pressure in the subject based on a calculation of timing of the one or more heartbeats relative to the timing of the first blood pressure pulse relative to the timing of the second blood pressure pulse.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 depicts a diagrammatic view of an aspect of a remote blood pressure measurement system.

FIG. 2 depicts a diagrammatic view of an aspect of a remote blood pressure measurement system.

FIG. 3 depicts a diagrammatic view of an aspect of a remote blood pressure measurement system.

FIG. 4 depicts a diagrammatic view of an aspect of a remote blood pressure measurement system.

FIG. 5 depicts a diagrammatic view of an aspect of a method for remotely determining blood pressure in a mammalian subject utilizing video magnification.

FIG. 6 depicts a diagrammatic view of an aspect of a remote blood pressure measurement system.

FIG. 7 depicts a diagrammatic view of an aspect of a method for remotely determining blood pressure in a mammalian subject.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Devices, systems, and methods are disclosed herein for remotely determining blood pressure in a mammalian subject. A method for remotely determining blood pressure in a mammalian subject is disclosed that includes emitting multiple micropower impulse radar (MIR) pulses from one or more radiation pulse generator transmitters to a first location on a skin surface of the mammalian subject; utilizing one or more contact-free tissue probes including one or more MIR pulse detectors responsive to scattered multiple MIR pulses in an environment of the mammalian subject; determining one or more heartbeats of the mammalian subject by detecting multiple MIR pulses scattered to the one or more MIR pulse detectors; detecting at one or more optical radiation detectors a first reflected optical radiation from blood at a second location at a skin surface of the subject and a second reflected optical radiation from blood at a third location at the skin surface of the subject; determining the timing of a first blood pressure pulse at the second location by comparing levels of the first reflected optical radiation measured at different times; determining the timing of a second blood pressure pulse at the third location by comparing levels of the second reflected optical radiation measured at different times; and determining the blood pressure in the subject based on a calculation of timing of the one or more heartbeats relative to timing of the first blood pressure pulse and the timing of the second blood pressure pulse. In some aspects, the method may further include utilizing the one or more radiation pulse generator transmitters and the one or more tissue probes mounted to an exterior surface in an environment of the subject. In some aspects, the method may further include utilizing the one or more radiation pulse generator transmitters and the one or more tissue probes mounted to an exterior surface in an environment of the subject.

A device for use in the method for remotely determining blood pressure in a mammalian subject is disclosed. A device is disclosed that includes one or more radiation pulse generator transmitters emitting multiple micropower impulse radar (MIR) pulses to a first location on a skin surface of the mammalian subject; one or more contact-free tissue probes including one or more MIR pulse detectors responsive to scattered multiple MIR pulses in an environment of the mammalian subject, wherein the scattered multiple MIR pulses to the one or more MIR pulse detectors are operable to measure one or more heart beats of the mammalian subject; one or more optical radiation detectors operable to measure a first reflected optical radiation from blood at a second location at a skin surface of the subject and a second reflected optical radiation from blood at a third location at the skin surface of the subject; a controller operable to determine the timing of a first blood pressure pulse at the second location by comparing levels of the first reflected optical radiation measured at different times and operable to determine the timing of a second blood pressure pulse at the third location by comparing levels of the second reflected optical radiation measured at different times; and operable to determine the blood pressure in the subject based on a calculation of timing of the one or more heartbeats relative to timing of the first blood pressure pulse and the timing of the second blood pressure pulse. The heart monitor emitting multiple radiation pulses from one or more radiation pulse generator transmitters includes, but is not limited to, a micro-impulse radar sensor, a remote EKG sensor, a wearable EKG sensor or a PMG sensor. In some aspects, the optical radiation includes, but is not limited to, ambient room radiation, ambient solar radiation, artificial room radiation, an optical light source, LED radiation, incandescent radiation, or fluorescent radiation. In some aspects, the optical radiation source includes, but is not limited to, an incoherent optical source. In some aspects, the optical radiation source includes, but is not limited to, a laser light source.

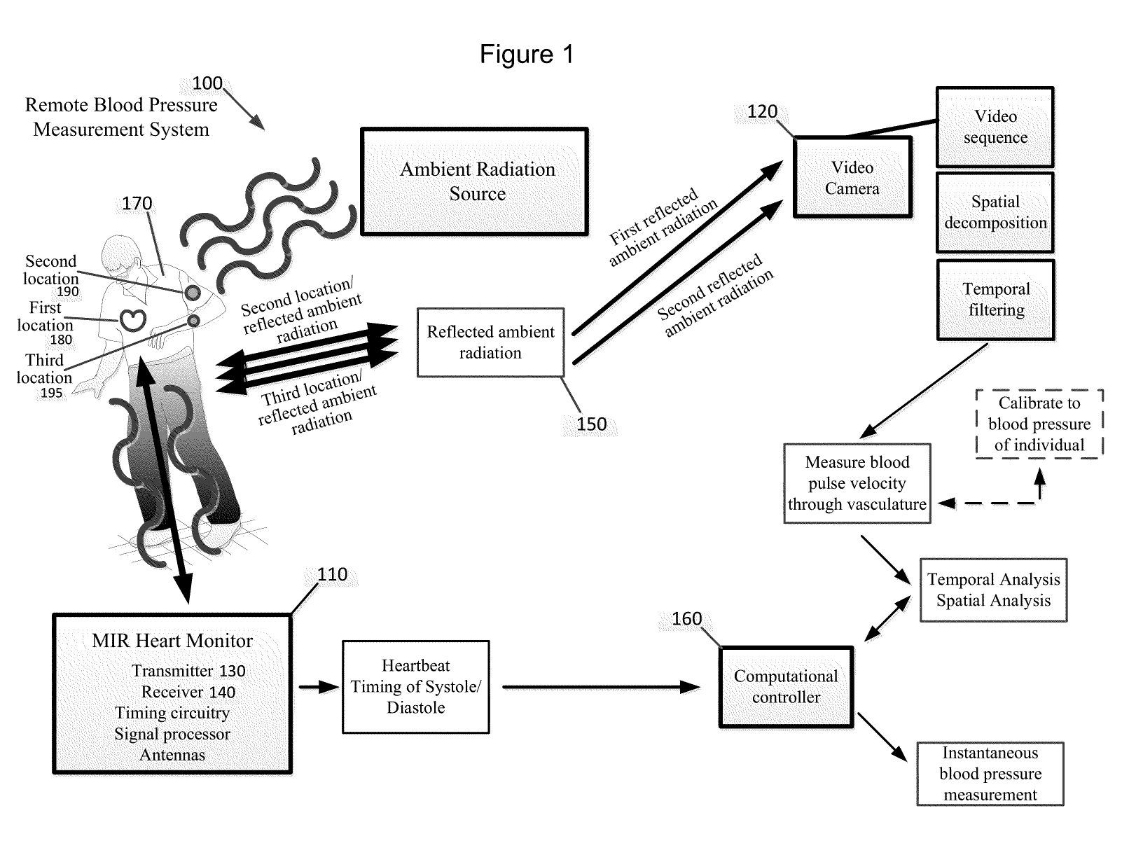

FIG. 1 depicts a diagrammatic view of an aspect of a remote blood pressure measurement system 100 including an MIR heart monitor 110 and a video camera 120. The remote blood pressure measurement system includes a device including one or more radiation pulse generator transmitters 130 emitting multiple micropower impulse radar (MIR) pulses to a first location 180 on a skin surface near a heart of the mammalian subject; one or more contact-free tissue probes including one or more MIR pulse detectors/receivers 140 responsive to reflected or scattered multiple MIR pulses in an environment of the mammalian subject, wherein the reflected or scattered multiple MIR pulses to the one or more MIR pulse detectors are operable to measure one or more heart beats of the mammalian subject 170; one or more optical radiation detectors 120, e.g., video camera, operable to measure a first reflected optical radiation 150 from blood at a second location 190 at a skin surface of the subject and a second reflected optical radiation 150 from blood at a third location 195 at the skin surface of the subject; and a controller 160 operable to determine timing of a first blood pressure pulse by comparing levels of the first reflected optical radiation measured at different times, operable to determine timing of a second blood pressure pulse by comparing levels of the second reflected optical radiation measured at different times, and operable to determine the blood pressure in the subject based on a comparison of the timing of the one or more heartbeats with the timing of the first blood pressure pulse and the second blood pressure pulse.

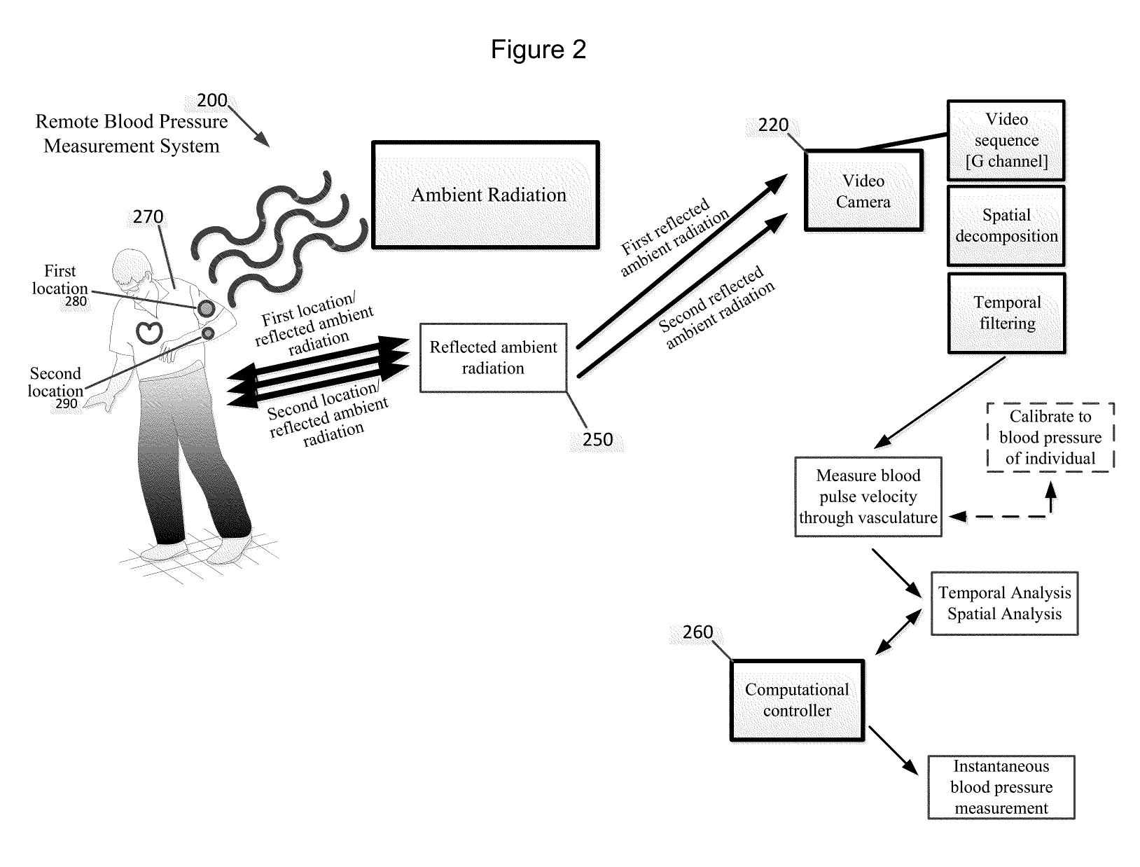

FIG. 2 depicts a diagrammatic view of an aspect of a remote blood pressure measurement system 200 including reflected optical radiation 250 to a video camera 220. A device includes one or more optical radiation detectors 220, e.g., video camera, operable to measure a first reflected optical radiation 250 from blood at a first location 280 at a skin surface of the subject 270 and a second reflected optical radiation 250 from blood at a second location 290 at the skin surface of the subject; and a controller 260 operable to determine timing of a first blood pressure pulse by comparing levels of the first reflected optical radiation measured at different times, operable to determine timing of a second blood pressure pulse by comparing levels of the second reflected optical radiation measured at different times, and operable to determine the blood pressure in the subject based on a comparison of the timing of the first blood pressure pulse and the second blood pressure pulse.

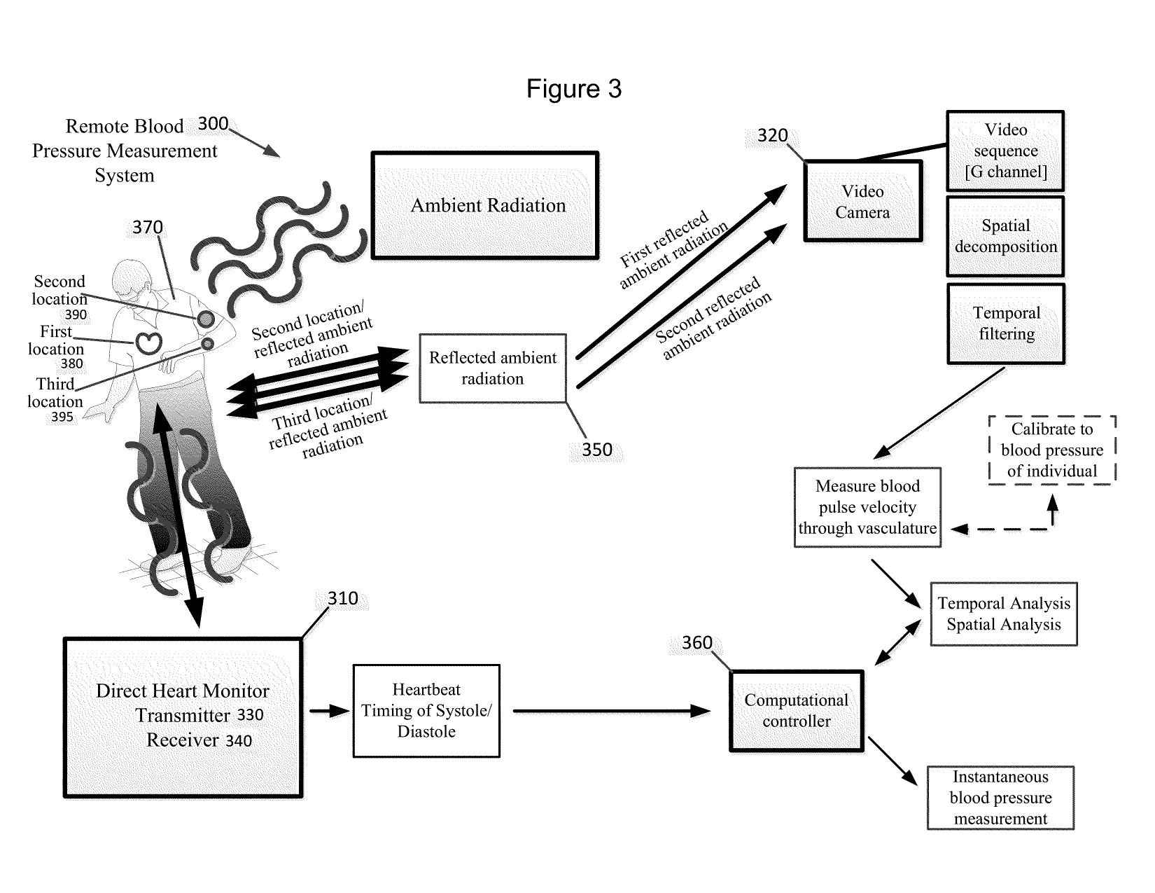

FIG. 3 depicts a diagrammatic view of an aspect of a remote blood pressure measurement system 300 including a heart monitor 310 and a video camera 320. A device includes: one or more radiation pulse generator transmitters 330 emitting multiple power pulses to a first location 380 on a skin surface near a heart of the mammalian subject; one or more tissue probes including one or more power pulse detectors/receivers 340 responsive to reflected power pulses in an environment of the mammalian subject, wherein the reflected multiple power pulses to the one or more power pulse detectors 340 are operable to measure one or more heart beats of the mammalian subject; one or more optical radiation detectors 320, e.g., video camera, operable to measure a first reflected optical radiation 350 from blood at a second location 390 at a skin surface of the subject and a second reflected optical radiation 350 from blood at a third location 395 at the skin surface of the subject; and a controller 360 operable to determine timing of a first blood pressure pulse by comparing levels of the first reflected optical radiation measured at different times, operable to determine timing of a second blood pressure pulse by comparing levels of the second reflected optical radiation measured at different times, and operable to determine the blood pressure in the subject based on a comparison of the timing of the one or more heartbeats with the timing of the first blood pressure pulse and the second blood pressure pulse.

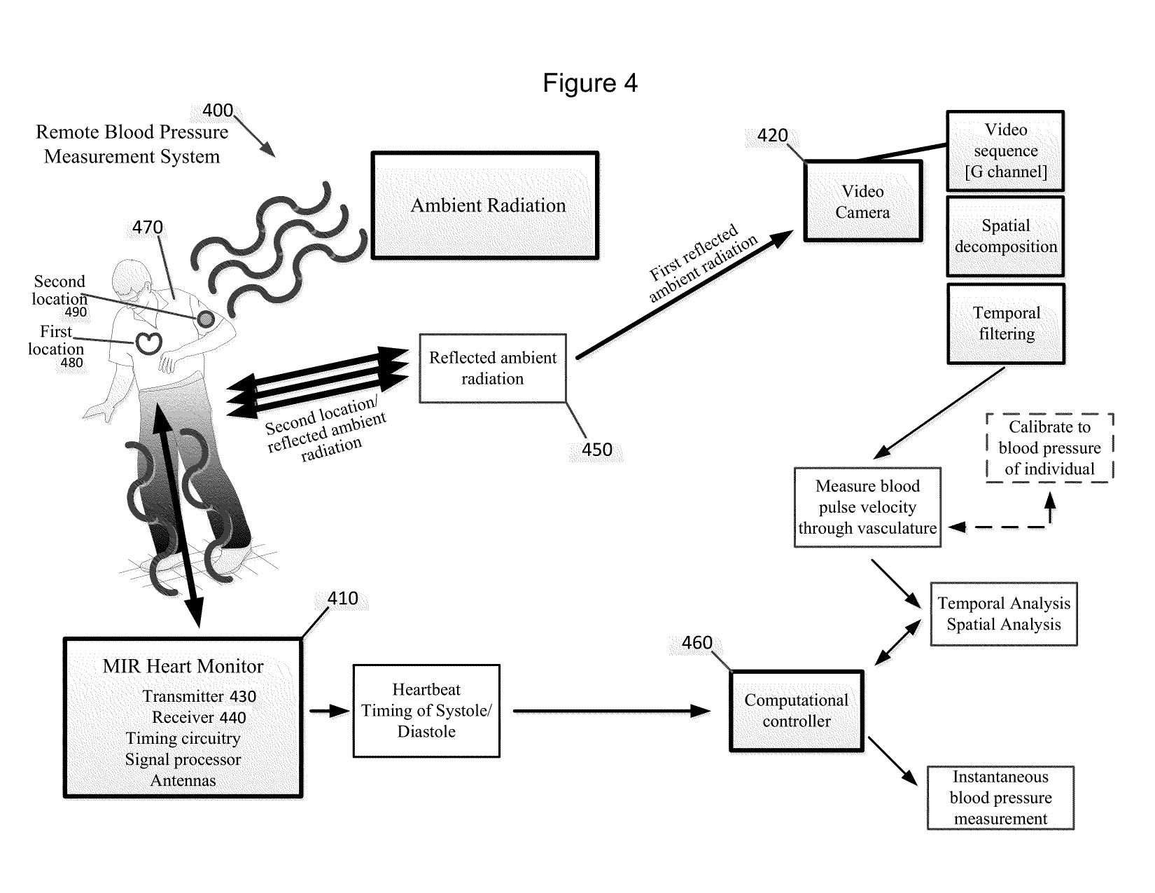

FIG. 4 depicts a diagrammatic view of an aspect of a remote blood pressure measurement system 400 including an MIR heart monitor 410 and a video camera 420. A device includes one or more radiation pulse generator transmitters 430 emitting multiple micropower impulse radar (MIR) pulses to a first location 480 on a skin surface near a heart of the mammalian subject 470; one or more contact-free tissue probes including one or more MIR pulse detectors/receivers 440 responsive to scattered multiple MIR pulses in an environment of the mammalian subject, wherein the scattered multiple MIR pulses to the one or more MIR pulse detectors are operable to measure one or more heart beats of the mammalian subject; one or more optical radiation detectors 420, e.g., video camera, operable to measure a first reflected optical radiation 450 from blood at a second location 490 at a skin surface of the subject; and a controller 460 operable to determine timing of a first blood pressure pulse by comparing levels of the scattered multiple MIR pulses measured at different times, operable to determine timing of a second blood pressure pulse by comparing levels of the first reflected optical radiation measured at different times, and operable to determine the blood pressure in the subject based on a comparison of the timing of the one or more heartbeats from the first blood pressure pulse with the timing of the second blood pressure pulse.

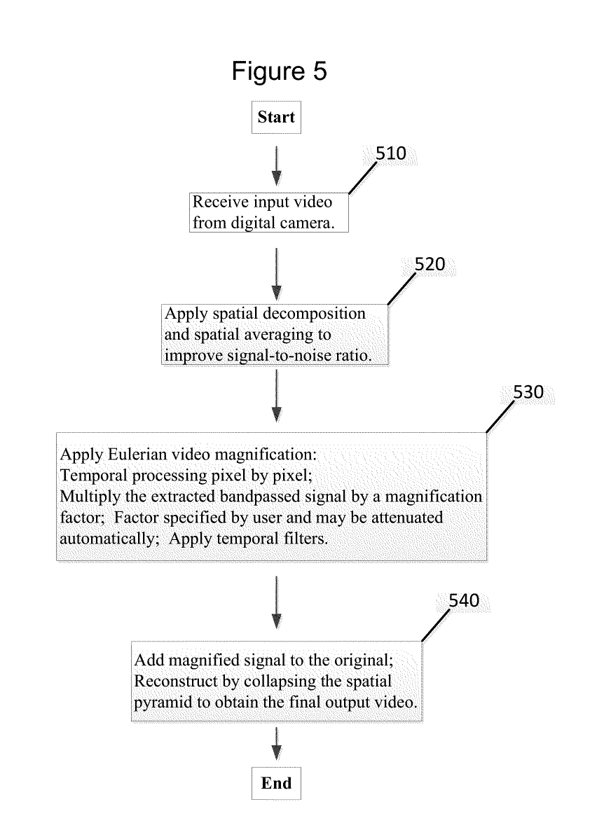

FIG. 5 depicts a diagrammatic view of an aspect of a method for remotely determining blood pressure in a mammalian subject utilizing video magnification. The video input from a video camera 510 is used to calculate timing of the reflected optical radiation, and operable to determine the blood pressure in the subject based on a comparison of the timing of the one or more heartbeats with the timing of a first reflected optical radiation and a second reflected optical radiation. Utilizing input video from a video camera 510, spatial decomposition and spatial averaging 520 are calculated to improve signal-to-noise ratio. From the data, Eulerian video magnification 530 provides temporal processing (pixel-wise) and multiplication of the extracted bandpassed signal by a magnification factor. The magnification factor may be specified by the user and may be attenuated automatically. Temporal processing involves the use of temporal filters. The magnified signal is added to the original 540. The final output is reconstructed by collapsing the spatial pyramid to obtain the final output video.

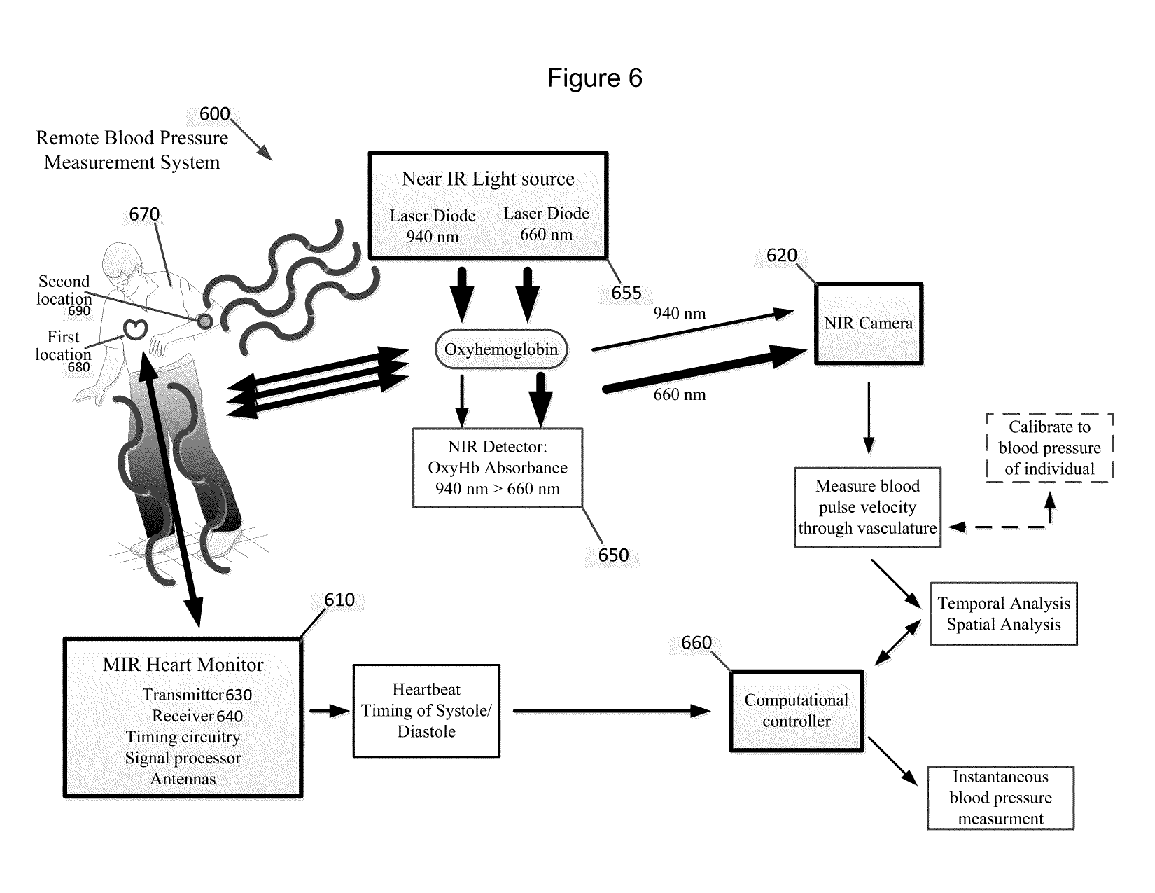

FIG. 6 depicts a diagrammatic view of an aspect of a remote blood pressure measurement system 600 including an MIR heart monitor 610 and a video camera 620. The remote blood pressure measurement system includes a device including one or more radiation pulse generator transmitters 630 emitting multiple micropower impulse radar (MIR) pulses to a first location 680 on a skin surface near a heart of the mammalian subject; one or more contact-free tissue probes including one or more MIR pulse detectors/receivers 640 responsive to scattered multiple MIR pulses in an environment of the mammalian subject 670, wherein the scattered multiple MIR pulses to the one or more MIR pulse detectors/receivers 640 are operable to measure one or more heart beats of the mammalian subject; one or more near infrared red (NIR) light sources 655 and one or more NIR radiation detectors 650 operable to measure a first reflected NIR radiation from blood at a second location 690 at a skin surface of the subject and measuring the first reflected NIR radiation at two or more wavelengths, e.g., 940 nm and 660 nm, to determine absorbance by oxyhemoglobin; and a controller 660 operable to determine timing of a first blood pressure pulse by comparing levels of the scattered multiple MIR pulses measured at different times, operable to determine timing of a second blood pressure pulse by comparing levels of the first reflected optical radiation measured at two or more wavelengths, e.g., 940 nm and 660 nm, and at different times, and operable to determine the blood pressure in the subject based on a comparison of the timing of the one or more heartbeats from the first blood pressure pulse with the timing of the second blood pressure pulse.

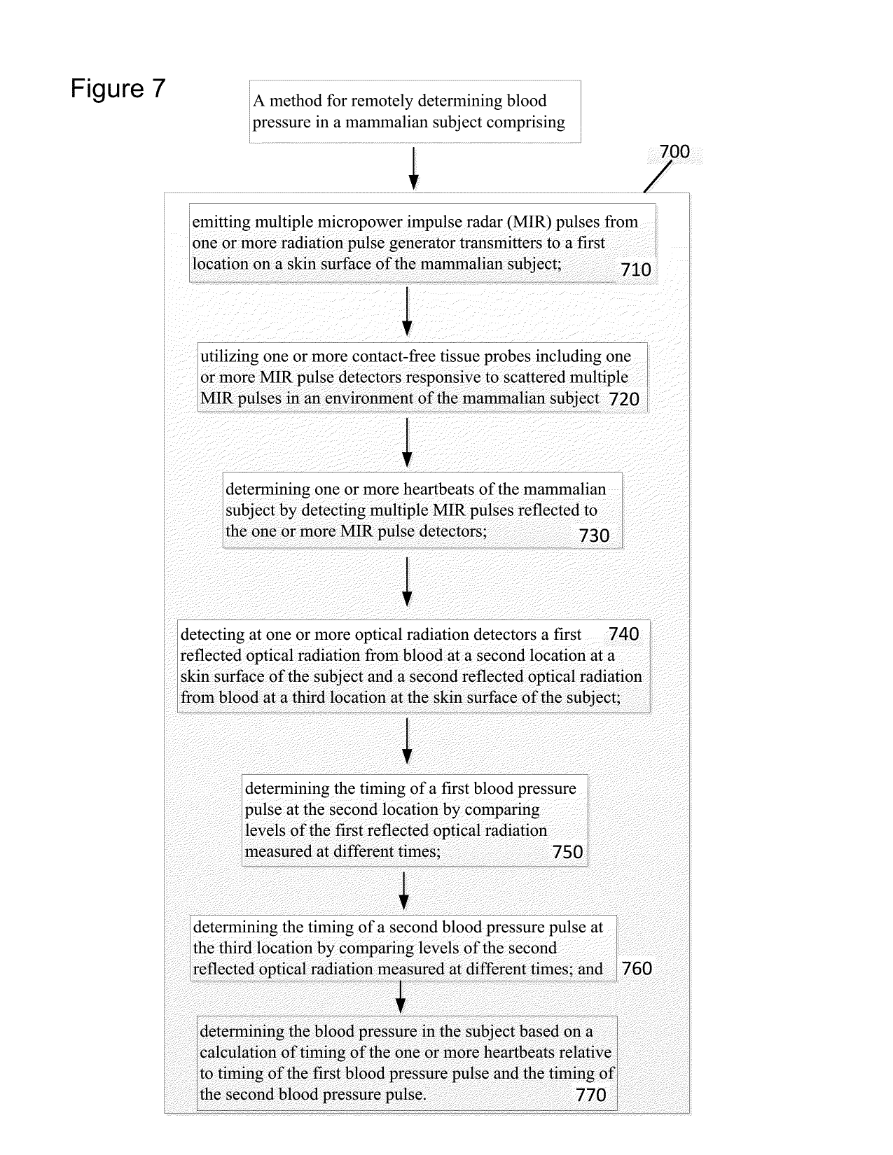

FIG. 7 depicts a diagrammatic view of an aspect of a method for remotely determining blood pressure in a mammalian subject 700 comprising: emitting 710 multiple micropower impulse radar (MIR) pulses from one or more radiation pulse generator transmitters to a first location on a skin surface of the mammalian subject; utilizing 720 one or more contact-free tissue probes including one or more MIR pulse detectors responsive to scattered multiple MIR pulses in an environment of the mammalian subject; determining 730 one or more heartbeats of the mammalian subject by detecting multiple MIR pulses scattered to the one or more MIR pulse detectors; detecting 740 at one or more optical radiation detectors a first reflected optical radiation from blood at a second location at a skin surface of the subject and a second reflected optical radiation from blood at a third location at the skin surface of the subject; determining 750 the timing of a first blood pressure pulse at the second location by comparing levels of the first reflected optical radiation measured at different times; determining 760 the timing of a second blood pressure pulse at the third location by comparing levels of the second reflected optical radiation measured at different times; and determining 770 the blood pressure in the subject based on a calculation of timing of the one or more heartbeats relative to timing of the first blood pressure pulse and the timing of the second blood pressure pulse.

### Methods Utilizing Optical Irradiation of Tissues and ECG to Measure Blood Pressure in a Subject

Non-implantable remote monitoring devices to monitor a patient's arterial blood pressure may be used. In some embodiments, the device can be configured to be located remote from the patient's skin. The device would include surface electrodes remote from the patient's skin so that a surface electrocardiogram (surface ECG) that is indicative of electrical activity of the patient's heart can be obtained. An arterial blood pressure monitor can be located within the device housing. In some embodiments, the surface ECG electrodes can be attached to a housing, e.g., substantially flush with and/or adjacent to the housing. In such embodiments, the housing can be located remote from the patient's skin. In other embodiments, the surface ECG electrodes can be remote from the housing and remote from the patient's skin, e.g., outside of a patient's rib cage.

In some embodiments, the radiation detector, e.g., ambient radiation detector, of the non-implantable monitoring device can be within, integral with or attached to the housing (e.g., a light source and a light detector can be within, integral with or attached to the housing). In such embodiments, the light source and the detector can face the patient's skin that is adjacent the housing. Alternatively, optical fibers can be used to transmit light produced by the light source to a portion of a patient's body that is remote from the housing, and can provide a portion of the transmitted light reflected from and/or transmitted through the portion of the patient's body to the radiation detector. In some embodiments, the radiation detector can includes a light source and a light detector that are located within, integral with or attached to the lead that extends from the housing to thereby enable the light source and light detector to be placed adjacent a portion of the patient's body, e.g., a finger, arm, or earlobe, that is remote from the device housing. See, e.g., U.S. Pat. No. 8,162,841, which is incorporated herein by reference.

### Methods Utilizing Optical Irradiation of Tissues and Magnified Video Images to Measure Blood Pressure in a Subject

Video images of the subject may be analyzed to determine blood pressure in the subject. The video images from two locations on the subject may be analyzed, e.g., regions of interest may be selected using a graphic user interface, or from a grid superimposed on the images. The images are analyzed temporally to calculate a blood pulse velocity. Videos are processed using a technique to reveal temporal variations that are difficult or impossible to see with the naked eye and display them in an indicative manner. Eulerian video magnification takes a standard video sequence as input, and applies spatial decomposition, followed by temporal filtering to the frames. The resulting signal is then amplified to reveal hidden information. Using this method, one may visualize the flow of blood as it fills the face and also to amplify and reveal small motions. The technique may run in real time to show phenomena occurring at temporal frequencies and through data analysis to determine blood pressure in the subject. See, e.g., Wu et al., ACM Trans. Graph. 31, 4, Article 65, July 2012; available online at http://doi.acm.org/10.1145/2185520.2185561, which is incorporated herein by reference.

The method for determining blood pressure in the subject takes a video of the subject as input and exaggerates subtle color changes and imperceptible motions. To amplify motion, the method magnifies temporal color changes using spatio-temporal processing. The Eulerian-based method, which temporally processes pixels in a fixed spatial region, reveals informative signals and amplifies small motions in real-world videos.

The human visual system has limited spatio-temporal sensitivity, but many signals that fall below this capacity can be informative. For example, human skin color varies slightly with blood circulation. This variation, while invisible to the naked eye, can be exploited to extract pulse rate. Similarly, motion with low spatial amplitude, while hard or impossible for humans to see, can be magnified to reveal interesting mechanical behavior.

A combination of spatial and temporal processing of videos may amplify subtle variations that reveal important aspects of the physical world. The method for measuring blood pressure in the subject considers the time series of color values at any spatial location (pixel) and amplify variation in a given temporal frequency band of interest. For example, one may automatically select, and then amplify, a band of temporal frequencies that includes plausible human heart rates. The amplification reveals the variation of redness as blood flows through the face. For this application, temporal filtering needs to be applied to lower spatial frequencies (spatial pooling) to allow such a subtle input signal to rise above the camera sensor and quantization noise.

The temporal filtering approach not only amplifies color variation, but can also reveal low-amplitude motion. In some aspects the approach provides a mathematical analysis to explain how temporal filtering interplays with spatial motion in videos. An analysis relies on a linear approximation related to the brightness constancy assumption used in optical flow formulations. Conditions under which this approximation holds may be derived. This leads to a multiscale approach to magnify motion without feature tracking or motion estimation.

The system for measuring blood pressure in the subject utilizes a Eulerian video magnification framework to first decompose the input video sequence into different spatial frequency bands and then to apply the same temporal filter to all bands. The filtered spatial bands are then amplified by a given factor, added back to the original signal, and collapsed to generate the output video. The choice of temporal filter and amplification factors can be tuned to support different applications.

The method and system combine spatial and temporal processing to emphasize subtle temporal changes in a video. The system as illustrated includes decomposing the video sequence into different spatial frequency bands. These bands might be magnified differently because (a) they might exhibit different signal-to-noise ratios or (b) they might contain spatial frequencies for which the linear approximation used in our motion magnification does not hold. In the latter case, the amplification for these bands is reduced to suppress artifacts. When the goal of spatial processing is simply to increase temporal signal-to-noise ratio by pooling multiple pixels, the frames of the video are spatially low-pass filtered and down-sampled for computational efficiency. In the general case, however, a full Laplacian pyramid is computed.

The system for measuring blood pressure in the subject utilizes the computational results generated using non-optimized MATLAB code on a machine with a six-core processor and 32 GB RAM. The computation time per video was on the order of a few minutes. A separable binomial filter of size five was used to construct the video pyramids. An application that allows users to reveal subtle changes in real-time from live video feeds serves as a microscope for temporal variations. It is implemented in C is entirely CPU-based, and processes 640 480 videos at 45 frames per second on a standard laptop.

Four steps to process an input video by Eulerian video magnification are provided: (1) select a temporal bandpass filter; (2) select an amplification factor, a; (3) select a spatial frequency cutoff (specified by spatial wavelength, λ_{c}) beyond which an attenuated version of is used; and (4) select the form of the attenuation for a—either force α to zero for all λ<λ_{c}, or linearly scale a down to zero. The frequency band of interest can be chosen automatically in some cases, but it is often important for users to be able to control the frequency band corresponding to their application. In a real-time application, the amplification factor and cutoff frequencies are all customizable by the user. See, e.g., Wu et al., ACM Trans. Graph. 31, 4, Article 65, July 2012; available online at http://doi.acm.org/10.1145/2185520.2185561, which is incorporated herein by reference.

### Methods Utilizing a Heart Monitor to Measure Heart Rate for Measuring Blood Pressure in a Subject

The system for measuring blood pressure in the subject may include a heart monitor to detect heartbeat and to measure heart rate and ECG. The heart monitor may be modified to include a transmitting antenna and a receiving antenna for remotely detecting heart and respiratory motion remotely and through various materials, e.g., a mattress pad, a chair back, or other object. The transmitting antenna and the receiving antenna of the heart monitor have been modified to permit greater scanning range. The heart monitor may have an audible output. A range control is provided, and can be set to detect heart beat and respiration at a distance of about 6 feet. See, e.g., U.S. Pat. No. 5,766,208, which is incorporated herein by reference.

The general operation of the heart monitor is also based on the emission of a pulse from a transmit antenna, waiting for a brief period of time, and then opening a gate connected to a receive antenna to allow the reflected pulse to be sampled. However, where the heart monitor is used as a non-contact cardiopulmonary monitor, the waiting period corresponds to 12 inches or more of round trip time of flight at the speed of light in free space, or in a combination of free space and one inch of tissue.

In the transmit path, the pulse repetition frequency/pulse repetition interval (PRF/PRI) generator drives an impulse generator, which provides a 5V 200 ps wide half-sine transmit pulse that is applied to a transmit antenna (T). A noise generator may modulate the PRF/PRI generator to create a PRF with a 1 MHz average and 1-10% random variation about 1 MHz, e.g., a 1-10% PRF dither. The electrical length of the transmit antenna is set to be short relative to the spectral content of the half-sine to avoid ringing.

A receive antenna picks up the pulse reflected from a body organ, such as a heart behind a chest wall, and applies it to a sample/hold (S/H) circuit that is gated by a gating pulse from a gating path. The gating pulse is delayed by approximately 3 nanoseconds from the time that the transmit antenna radiates the pulse. Therefore, reflections occurring at about 12 inches from the transmit and receive antennas are thereby sampled. Pulses from the PRF/PRI generator which are input into the transmit path are simultaneously input into the gating path where they pass through an range delay generator followed by an impulse generator, which produces a 200 ps wide gating pulse for controlling a gating switch.

The timing relationship may be represented as four waveforms over a one pulse repetition interval (PRI). A 200 ps wide impulse is radiated from the transmit antenna. The reflected impulse from the receive antenna coincides with the gating pulse. Each received pulse produces an incremental voltage change ΔV on the capacitor of the S/H circuit. The capacitor voltage is the output of the averaging S/H circuit. The increment ΔV=1/N of the total received pulse, where N is the number of samples averaged, typically about 10,000. It should be understood that N can assume a different value.

In the receive path, the output of the summation element is amplified by the amplifier, typically 70 dB across a passband of 0.05-10 Hz, and applied, optionally, to cardiac and pulmonary bandpass filters, respectively. See, e.g., U.S. Pat. No. 5,766,208, which is incorporated herein by reference.

### Pulse Wave Velocity (PWV)-Based Methods for Blood Pressure Measurement

A method for remotely monitoring blood pressure includes estimating the pulse wave velocity (PWV) using a circulatory waveform signal measured from one or more remote sensors. The remote sensors measure blood pulse data from two regions of interest (ROI) separated by a known distance that are selected from the video frames using a grid overlay and a graphical user interface. For example, two regions of interest (ROI) may include the wrist on the ulnar artery and the smallest finger on the digital artery. Blood pressure (BP) can be derived from the estimation of the pulse wave velocity (PWV). In particular, the mapping between PWV and BP is based on the relationship that each of them shares with arterial vessel elasticity (Moens-Korteweg equation). A calibration procedure may be defined to individually calibrate the measured PWV to peripheral BP using hydrostatic pressure variation. See e.g., Fortino and Giampá, Medical Measurements and Applications Proceedings (MeMeA), 2010 IEEE International Workshop, Apr. 30 to May 1, 2010 Ottawa, Canada, which is incorporated herein by reference.

In some embodiments, a method for remotely monitoring blood pressure includes analyzing the relationship between BP and the characteristics of the pulse wave velocity (PWV) signal by processing the PWV signal at two regions of interest (ROI) separated by a known distance. For example, an approach may be based on the extraction of specific temporal characteristics of the PWV signal and a subsequent BP estimation based on a linear model containing considered characteristics as parameters. The considered characteristics include:
Width1, is the width of the signal at ⅔ of the amplitude peak-to-peak;
Width2, is the width of the signal at ½ of the amplitude peak-to-peak;
T_{S}, is the Systolic time defined as the ascending time of the signal from its minimum to its maximum;
T_{D}, is the Diastolic time, defined as the descendent time of the signal from its maximum to its minimum.

On the basis of tests on subjects that are carried out under different load conditions (rest, step-climbing, rest after step-climbing), the diastolic time (T_{D}) is correlated to BP. The cardiovascular parameters and the PWV signal may be correlated for different subjects to correct for variation that can affect the estimation of the parameter under measurement. In particular, the operational approach consists in the acquisition of the PWV signal from the forefinger and the exclusive use of T_{D} for BP estimation. T_{D} can be computed through a peak detection algorithm by determining the time instants in which first the maximum peak and then the minimum peak are detected. The identification can be done through a minimum square method applied to the real values of BP and to the corresponding values of the characteristic T_{D}. The functions to be estimated are two (one for the DBP and the other for SBP):

SBP=αSBP·T_{D}+bSBP

DBP=αDBP·T_{D}+bDBP

The identification of the coefficients of each function should be independently determined. Once identified the model, DBM and SBP can be obtained by computing T_{D}. See e.g., U.S. Pat. No. 7,608,045 issued to Mills on Oct. 27, 2009; Teng and Zhang, Proceedings 25^{th} Annual International Conf. IEEE EMBS, pp. 3153-3156, Sep. 17-21, 2003 and Fortino and Giampá, Medical Measurements and Applications Proceedings (MeMeA), 2010 IEEE International Workshop, Apr. 30 to May 1, 2010 Ottawa, Canada, which are incorporated herein by reference.

### PROPHETIC EXEMPLARY EMBODIMENTS

### Example 1

### Method to Remotely Monitor Blood Pressure Using Video Magnification and Ambient Light

A patient with heart disease is monitored with a remote blood pressure monitoring system. The system includes a monocular video camera, a computer/controller, and methods to magnify blood pulses in the vasculature and to remotely determine blood pressure. A video camera, Dropcam, with 1280×720 pixel resolution and a frame speed of 30 frames per second (see e.g., a Dropcam Technical Specs Sheet available from Dropcam HQ, San Francisco, Calif.) is mounted on the ceiling or wall over the patient's bed and focused on the forehead of the patient using the 4× digital zoom. Digital images are transferred to a computer/controller for spatial and temporal processing to magnify the pulsing of blood in the vasculature underlying the dermis of the patient's forehead. Methods to amplify the movement of swelling vasculature and to remotely visualize the color changes due to blood pulsing are described (see e.g., Wu et al., ACM Trans. Graph. 31, 4, Article 65, July 2012; available online at http://doi.acm.org/10.1145/2185520.2185561, which is incorporated herein by reference). In order to calculate blood pressure from blood pulse data, two regions of interest (ROI) separated by a known distance are selected from the video frames using a grid overlay and a graphical user interface (see e.g., Verkruysse et al., Optics Express 16: 21434-21445, 2008 which is incorporated herein by reference). The selected regions are analyzed temporally and magnified to detect reflected ambient light at specific wavebands. For example green bands are absorbed by hemoglobin in the blood and reveal pulsing of blood by a reduction in reflected light when blood vessels swell. Also specific wavelengths of light are absorbed preferentially by oxyhemoglobin versus reduced hemoglobin (e.g., 940 nm versus 660 nm respectively). Moreover the departure and arrival times of blood pulses between the two ROI are determined and used to calculate blood pulse velocity. Methods to calculate the blood pulse transfer time and blood pulse velocity are described (see e.g., Verkruysse et al., Ibid.). The locations of the ROI and corresponding blood pulses relative to the heart are used to calculate hydrostatic pressure and ultimately the patient's correct blood pressure. Blood pressure (e.g., systolic pressure, diastolic pressure, or average pressure) can be calculated from blood pulse data. See e.g., U.S. Pat. No. 7,608,045 issued to Mills on Oct. 27, 2009; Teng and Zhang, Proceedings 25^{th} Annual International Conf IEEE EMBS, pp. 3153-3156, Sep. 17-21, 2003 and Fortino and Giampá, Medical Measurements and Applications Proceedings (MeMeA), 2010 IEEE International Workshop, Apr. 30 to May 1, 2010 Ottawa, Canada, which are incorporated herein by reference. These relationships can be expressed as functions which ultimately relate the measured difference in pulse timings, Δt, between the two ROI to the inferred blood pressure, P. The functions can be expressed either as P=P1(Δt) or as its inverse, Δt=T1(P). These allow us to directly solve for P=P1(Δt) given a measured value for Δt, or to implicitly solve for the P value using Δt=T1(P). Slightly different P1(Δt) or T1(P) functions e.g., with different parameter values) can be used depending upon the blood pressure metric used (e.g., systolic pressure, diastolic pressure, or average pressure).

The remote blood pressure measuring system is initialized and calibrated when the patient enters the hospital bed. Video images of the patient's face are transmitted to an electronic health record to identify the patient and document their arrival in the hospital bed. Initially the system remotely monitors blood pressure while simultaneously a standard blood pressure cuff is used to measure blood pressure. The system correlates remotely determined blood pressure values with cuff-derived blood pressure values to calibrate the remote blood pressure measuring system. Methods to calibrate blood pulse-derived pressures using traditional blood pressure measurements are described (see e.g., Beiderman et al., J. Biomedical Optics 15: 0061707-1-0061707-7, 2010 and U.S. Pat. No. 7,544,168 issued to Nitzan on Jun. 9, 2009 which are incorporated herein by reference). Once calibrated the remote blood pressure measuring system continuously reports systolic and diastolic blood pressure to an electronic health record and to the patient's caregivers. Abnormal blood pressure readings are detected by the system and an alert is sent to the patient's caregivers. Cumulative blood pressure data stored in the patient's electronic record may be used to determine the schedule and dosage of medication, e.g., blood pressure medication, and to adjust the dosage and schedule as required based on the ongoing blood pressure readings.

### Example 2

### Method to Remotely Monitor Blood Pressure Using Video Magnification and Ambient Light for Hospitals

A hospital uses a remote blood pressure measuring system to monitor patients in their beds. The system uses ambient light and a NIR camera to detect blood pulses in the ulnar artery of the patients while in bed. Also a micro-impulse radar (MIR) source/detector is focused on the heart to detect heartbeat, systole and diastole. A system computer performs temporal and spatial analysis of the NIR and MIR images to determine blood pulse movement, heartbeat and calculates the patient's blood pressure. The remote blood pressure monitoring system is initialized and calibrated when the patient is admitted to the hospital room.

The remote system, including the NIR and MIR cameras are located on the ceiling over the patient's bed. The NIR camera is focused on the ulnar artery adjacent to the wrist. Alignment of the NIR camera with a tattoo or marking on the patients arm may be used to insure reproducible alignment with the ulnar artery on the wrist. A high speed camera is focused on the patient's ulnar artery adjacent to the wrist and captures and digitally stores images for temporal and spatial analysis. For example, a NIR camera with a frame rate of approximately 1700 Hz, high resolution (640×512 pixels), and a spectrum of 400 nm to 1700 nm is available from Xenics, Leuven, Belgium (see e.g., NIR camera spec. sheet available online at: http://www.xenics.com/en/index.asp which is incorporated herein by reference). The digital images may be processed to magnify the pulsing of blood through the ulnar artery (see e.g., Wu et al., ACM Trans. Graph. 31, 4, Article 65, July 2012; available online at http://doi.org/10.1145/2185520.2185561 which is incorporated herein by reference). The timing and location of blood pulses in the ulnar artery are matched with data on the contraction of the patient's heart which are obtained using a MIR system.

A MIR system including a transmitter, a receiver, timing circuitry, a signal processor and antenna is mounted on the ceiling or wall near the patient and the transmitter and receiver are focused on the patient's heart. (MIR pulse generators are available from Picosecond Pulse Labs, Boulder, Colo. 80301, USA.) MIR systems that can monitor heart muscle contractions are described (see e.g., U.S. Pat. No. 5,766,208 issued to McEwan on Jun. 16, 1998; and Azevedo S. G., McEwan T. E., “Micropower Impulse Radar,” Science and Technology Review, January/February 1996 pp. 17-29; available online at https://www.llnl.gov/str/pdfs/01_{—}96.2.pdf which are incorporated herein by reference). The timing of systole (ventricular contraction) and diastole are captured by MIR and transmitted to the system computer. Combining data on the timing of systole and diastole with data on the timing and location of the blood pulses in the ulnar artery allows calculation of blood pulse velocity and blood pressure (see e.g., U.S. Pat. No. 7,608,045 Ibid. and U.S. Patent Application No. 2009/0163821 by Sola I Caros et al. published on Jun. 25, 2009 which are incorporated herein by reference). To calibrate the system initial measurements are made simultaneously with a standard blood pressure cuff and the remote blood pressure system to correlate blood pressure with that determined from blood pulse velocities and to make corrections for hydrostatic pressure. Linear equations to calculate systolic and diastolic blood pressure with patient-specific coefficients are described (see Teng and Zhang, Ibid. and Fortino and Giampá, Ibid.). Once a patient's coefficients are derived from the calibration measurements, blood pressure (e.g., systolic pressure, diastolic pressure, or average pressure) can be calculated continuously based on blood pulse parameters. Moreover alternative methods to calibrate blood pulse-derived pressures using traditional blood pressure measurements are described (see e.g., Beiderman et al., J. Biomedical Optics 15: 0061707-1-0061707-7, 2010; U.S. Pat. No. 7,544,168 issued to Nitzan on Jun. 9, 2009 and U.S. Pat. No. 7,608,045 Ibid. which are incorporated herein by reference). These relationships can be expressed as functions which ultimately relate the measured difference in pulse timings, Δt, between the timing of the pulse at the ROI and the timing at the heart, to the inferred blood pressure, P. The functions can be expressed either as P=P2(Δt) or as its inverse, Δt=T2(P). These allow us to directly solve for P=P2(Δt) given a measured value for Δt, or to implicitly solve for the P value using Δt=T2(P). Slightly different P2(Δt) or T2(P) functions (e.g., with different parameter values) can be used depending upon the blood pressure metric used (e.g., systolic pressure, diastolic pressure, or average pressure).

The patient's calibration data and coefficients are stored in the remote blood pressure measuring system and used to continuously calculate blood pressure based on reflected ambient radiation from the patient's ulnar artery as detected by the NIR camera. The system automatically selects the same ROIs separated by the same distance used for calibration and computes the blood pressure using the coefficients established during calibration. Cumulative blood pressure data is transmitted to the patient's EHR and caregivers are alerted to blood pressure readings significantly outside the normal range.

### Example 3

### Device and Method for Remote Determination of Blood Pressure

A patient in an intensive care unit is monitored with a remote blood pressure measuring system. The system uses near infrared (NIR) light and a NIR camera to detect blood pulses in the skin. Also micro-impulse radar (MIR) is used to detect heartbeat, specifically systole and diastole. The system computer performs temporal and spatial analysis of the NIR camera images to determine pulse blood flow and combines heartbeat data to infer the patient's blood pressure.

A remote blood pressure measuring system is mounted on the wall or ceiling proximal to the patient's bed to monitor the patient's blood pressure continuously and transmit the cumulative blood pressure data to the patient's caregivers. The system includes a laser diode to irradiate the patient's face with a NIR beam. Laser diodes emitting NIR wavelengths are available from Axcel Photonics, Inc., Marlborough, Mass. For example, a laser diode which emits approximately 940 nm wavelength light is used since 940 nm light is preferentially absorbed by oxyhemoglobin present in the blood, and generates an optical signature for a pulse of blood flowing in an artery or vein. A second laser diode emitting light at a different wavelength, e.g., 660 nm, may be used to generate a unique optical signature since oxyhemoglobin absorbs less light at 660 nm relative to hemoglobin. Optical signatures can be used to track pulses of blood in vivo as they flow through the vasculature. Methods and calculations to measure blood flow by irradiating tissues, termed photoplethysmography (PPG), are described (see e.g., Verkruysse et al., Optics Express 16: 21434-21445, 2008 and U.S. Pat. No. 8,162,841 issued to Keel et al. on Apr. 24, 2012 which are incorporated herein by reference.)

A NIR camera is mounted alongside the laser diodes to detect NIR light reflected from the patient's face. The high speed camera is focused on the patient's face and captures and digitally stores images for temporal and spatial analysis. For example, a NIR camera with a frame rate of approximately 1700 Hz, high resolution (640×512 pixels), and a spectral band of 400 nm to 1700 nm is available from Xenics, Leuven, Belgium (see e.g., NIR camera spec. sheet available online at: http://www.xenics.com/en/index.asp which is incorporated herein by reference). Computational methods to magnify video images and detect blood pulses in subcutaneous blood vessels are used to process the NIR images and to determine blood pulse movement (see e.g., Wu et al., ACM Trans. Graph. 31, 4, Article 65, July 2012; available online at http://doi.acm.org/10.1145/2185520.2185561 which is incorporated herein by reference). Video images from two locations on the face may be analyzed (e.g., regions of interest may be selected using a graphic user interface, or from a grid superimposed on the images; see Verkruysse et al., Ibid.). The images are analyzed temporally to calculate a blood pulse velocity (see e.g., Wu et al., Ibid.; U.S. Pat. No. 7,608,045 issued to Mills on Oct. 27, 2009 and U.S. Patent Application No. 2009/0163821 by Sola I Caros et al. published on Jun. 25, 2009 which are incorporated herein by reference). The regions of interest may coincide with regions of interest previously used for blood pressure calibration, as discussed below.

A MIR heart monitor is mounted with the NIR camera to monitor the heartbeat of the patient. A MIR system including a transmitter, a receiver, timing circuitry, a signal processor and antennas is mounted on the ceiling or wall near the patient and the transmitter and receiver are focused on the patient's heart. (MIR pulse generators (i.e., transmitters) are available from Picosecond Pulse Labs, Boulder, Colo. 80301, USA.) MIR systems that can monitor heart muscle contractions are described (see e.g., U.S. Pat. No. 5,766,208 issued to McEwan on Jun. 16, 1998; and Azevedo S. G., McEwan T. E., “Micropower Impulse Radar,” Science and Technology Review, January/February 1996 pp. 17-29; available online at https://www.llnl.gov/str/pdfs/01_{—}96.2.pdf which are incorporated herein by reference). The timing of systole (ventricular contraction) and diastole are captured by MIR and transmitted to the system computer. Combining data on the timing of systole and diastole with data on the timing and location of distal blood pulses (e.g., on the forehead) allows calculation of blood pressure (see e.g., U.S. Pat. No. 8,162,841, Ibid.). Also the location of the blood pulses (e.g., on the forehead) relative to the heart is used to calculate intravascular hydrostatic pressure and ultimately the patient's correct blood pressure. Methods for calculation of hydrostatic pressure and correcting blood pressure measurements are described (see e.g., U.S. Pat. No. 7,608,045 Ibid.).

Blood pressure values can be calculated from either the blood pulse velocity determined from NIR measurements at two regions of interest (referred to below as the NIR-NIR method) or from the timing difference between the MIR-derived start of a heartbeat and the NIR measured pulse arrival at a region of interest (referred to below as the MIR-NIR method). Alternatively, blood pressure values can be calculated by combining values obtained by each method.

To further elaborate these methods, let T1(P) be a predetermined function relating the NIR-NIR timing difference to blood pressure (discussed in Example 1), while T2(P) is a predetermined function relating the MIR-NIR timing difference to blood pressure (discussed in Example 2). Suppose the measured time differences are t1 and t2 respectively. In a pure NIR-NIR method, one calculates a blood pressure value P1 by solving the equation, t1=T1(P1) for P1. In a pure MIR-NIR method, one calculates a blood pressure value P2 by solving the equation, t2=T2(P2) for P2. If one has access to both NIR-NIR and MIR-NIR measurements, it is often the case that P1 and P2 are close to each other, but not precisely equal. We can form an improved blood pressure estimate, P, by combining both methods. One combination technique can be to use the numerical average of the two pressure values, i.e., P=0.5(P1+P2). Another combination technique can be to determine the pressure value which minimizes timing inconsistencies between the two measurement methods. Here, we select P so as to minimize the function [T1(P)−t1]̂2+[T2(P)−t2]̂2. It is clear that other combination techniques (e.g., giving more weight to one technique than the other) can be used to combine NIR-NIR and MIR-NIR measurements so as to determine improved blood pressure values.

The remote blood pressure measuring system is initialized and calibrated when the patient enters the ICU. Images from the NIR camera are used to identify the patient and transmitted to an electronic health record and the NIR source and camera are focused on the patient's forehead. Initially NIR imaging and MIR heart monitoring are used to monitor blood pressure while a standard blood pressure cuff is used to simultaneously measure blood pressure. The system correlates the remotely determined blood pressure values with the cuff-derived blood pressure values to calibrate the remote blood pressure measuring system. Methods to calibrate blood pressure measuring systems by correlating blood pulse parameters with standard cuff blood pressure measurements are described (see e.g., Beiderman et al., J. Biomedical Optics 15: 0061707-1-0061707-7, 2010 and U.S. Pat. No. 7,544,168 issued to Nitzan on Jun. 9, 2009 which are incorporated herein by reference). Once calibrated the remote blood pressure measuring system continuously reports systolic and diastolic blood pressure to the patient's electronic health record and to the patient's caregivers. Abnormal blood pressure values are recognized by the system computer and a warning may be sounded and a caregiver may be paged.

Each recited range includes all combinations and sub-combinations of ranges, as well as specific numerals contained therein.

All publications and patent applications cited in this specification are herein incorporated by reference to the extent not inconsistent with the description herein and for all purposes as if each individual publication or patent application were specifically and individually indicated to be incorporated by reference for all purposes.

Those having ordinary skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having ordinary skill in the art will recognize that there are various vehicles by which processes and/or systems and/or other technologies disclosed herein can be effected (e.g., hardware, software, and/or firmware), and that the preferred vehicle will vary with the context in which the processes and/or systems and/or other technologies are deployed. For example, if a surgeon determines that speed and accuracy are paramount, the surgeon may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies disclosed herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those having ordinary skill in the art will recognize that optical aspects of implementations will typically employ optically-oriented hardware, software, and or firmware.

In a general sense the various aspects disclosed herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices disclosed herein, or a microdigital processing unit configured by a computer program which at least partially carries out processes and/or devices disclosed herein), electrical circuitry forming a memory device (e.g., forms of random access memory), and/or electrical circuitry forming a communications device (e.g., a modem, communications switch, or optical-electrical equipment). The subject matter disclosed herein may be implemented in an analog or digital fashion or some combination thereof.

At least a portion of the devices and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

The herein described components (e.g., steps), devices, and objects and the description accompanying them are used as examples for the sake of conceptual clarity and that various configuration modifications using the disclosure provided herein are within the skill of those in the art. Consequently, as used herein, the specific examples set forth and the accompanying description are intended to be representative of their more general classes. In general, use of any specific example herein is also intended to be representative of its class, and the non-inclusion of such specific components (e.g., steps), devices, and objects herein should not be taken as indicating that limitation is desired.

With respect to the use of substantially any plural or singular terms herein, the reader can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an”; the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for remotely determining blood pressure in a mammalian subject comprising:
emitting multiple micropower impulse radar (MIR) pulses from one or more radiation pulse generator transmitters to a first location on a skin surface of the mammalian subject;
utilizing one or more contact-free tissue probes including one or more MIR pulse detectors responsive to scattered multiple MIR pulses in an environment of the mammalian subject;
determining one or more heartbeats of the mammalian subject by detecting multiple MIR pulses scattered to the one or more MIR pulse detectors;
detecting at one or more optical radiation detectors a first reflected optical radiation from blood at a second location at a skin surface of the subject and a second reflected optical radiation from blood at a third location at the skin surface of the subject;
determining the timing of a first blood pressure pulse at the second location by comparing levels of the first reflected optical radiation measured at different times;
determining the timing of a second blood pressure pulse at the third location by comparing levels of the second reflected optical radiation measured at different times; and
determining the blood pressure in the subject based on a calculation of timing of the one or more heartbeats relative to timing of the first blood pressure pulse and the timing of the second blood pressure pulse.

2. The method of claim 1, wherein an origin of the first blood pressure pulse and the second blood pressure pulse are a single origin.

3. The method of claim 1, comprising directing a first optical light source toward the second location for generation of the first reflected optical radiation.

4. The method of claim 1, comprising directing a second optical light source toward the third location for generation of the second reflected optical radiation.

5. (canceled)

6. The method of claim 1, wherein the first and second reflected optical radiation are two or more wavelengths of infrared radiation.

7. The method of claim 1, wherein the optical radiation comprises ambient room radiation, ambient solar radiation, artificial room radiation, radiation from an optical light source, LED radiation, incandescent radiation, or fluorescent radiation.

8. The method of claim 7, wherein the optical light source comprises an incoherent optical source.

9. The method of claim 1, comprising detecting the first and second reflected optical radiation from the second location and the third location at two or more time points.

10.-11. (canceled)

12. The method of claim 1, comprising measuring systole and diastole at the heart of the subject with the scattered multiple MIR pulses to the one or more MIR pulse detectors.

13.-14. (canceled)

15. The method of claim 1, comprising identifying the second location and the third location relative to the first location of the heart of the subject, and applying a corrected calculation to determine the blood pressure in the subject.

16. (canceled)

17. The method of claim 1, comprising utilizing the tissue probe and the one or more optical radiation detectors mounted to an exterior surface in the environment of the mammalian subject.

18.-47. (canceled)

48. A method for remotely determining blood pressure in a mammalian subject comprising:
emitting multiple micropower impulse radar (MIR) pulses from one or more radiation pulse generator transmitters to a first location on a skin surface of the mammalian subject;
utilizing one or more contact-free tissue probes including one or more MIR pulse detectors responsive to scattered multiple MIR pulses in an environment of the mammalian subject;
determining one or more heartbeats of the mammalian subject by detecting multiple MIR pulses scattered to the one or more MIR pulse detectors;
detecting at one or more optical radiation detectors a first reflected optical radiation from blood at a second location at a skin surface of the subject;
determining the timing of a first blood pressure pulse at the second location by comparing levels of the first reflected optical radiation measured at different times;
determining the blood pressure in the subject based on a calculation of the timing of the one or more heartbeats relative to the timing of the first blood pressure pulse.

49. The method of claim 48, comprising directing a first optical light source toward the second location for generation of the first reflected optical radiation.

50. The method of claim 48, wherein the first reflected optical radiation is infrared radiation.

51. The method of claim 48, wherein the optical radiation comprises ambient room radiation, ambient solar radiation, artificial room radiation, radiation from an optical light source, LED radiation, incandescent radiation, or fluorescent radiation.

52. (canceled)

53. The method of claim 48, wherein the optical light source comprises an incoherent optical source.

54. The method of claim 48, comprising detecting the MIR pulse from the first location and the first reflected optical radiation from the second location at two or more time points.

55. The method of claim 48, comprising detecting the first reflected optical radiation from the second location with a high frame rate camera.

56. The method of claim 55, comprising enhancing video resolution from the high frame rate camera.

57. The method of claim 48, comprising measuring systole and diastole at the heart of the subject with the scattered multiple MIR pulses to the one or more MIR pulse detectors.

58.-59. (canceled)

60. The method of claim 48, comprising identifying the second location relative to the first location of the heart of the subject, and applying a corrected calculation to determine the blood pressure in the subject.

61. (canceled)

62. The method of claim 48, comprising utilizing the tissue probe and the one or more optical radiation detectors mounted to an exterior surface in the environment of the mammalian subject.

63.-73. (canceled)

74. A method for remotely determining blood pressure in a mammalian subject comprising:
utilizing one or more contact-free tissue probes including one or more optical radiation detectors responsive to reflected optical radiation in an environment of the mammalian subject;
detecting at the one or more optical radiation detectors a first reflected optical radiation from blood at a first location at a skin surface of the subject and a second reflected optical radiation from blood at a second location at the skin surface of the subject;
determining the timing of a first blood pressure pulse at the first location by comparing levels of the first reflected optical radiation measured at different times;
determining the timing of a second blood pressure pulse at the second location by comparing levels of the second reflected optical radiation measured at different times; and
determining the blood pressure in the subject based on a calculation of the timing of the first blood pressure pulse relative to the timing of the second blood pressure pulse.

75. The method of claim 74, wherein an origin of the first blood pressure pulse and the second blood pressure pulse are a single origin.

76. The method of claim 74, comprising directing a first optical light source toward the first location for generation of the first reflected optical radiation.

77. The method of claim 74, comprising directing a second optical light source toward the second location for generation of the second reflected optical radiation.

78. The method of claim 74, wherein the first and second reflected optical radiation are infrared radiation.

79. The method of claim 78, wherein the first and second reflected optical radiation are two or more wavelengths of infrared radiation.

80. The method of claim 74, wherein the optical radiation comprises ambient room radiation, ambient solar radiation, artificial room radiation, radiation from an optical light source, LED radiation, incandescent radiation, or fluorescent radiation.

81. (canceled)

82. The method of claim 74, wherein the optical light source comprises an incoherent optical source.

83. The method of claim 74, comprising detecting the first and second reflected optical radiation from the first location and the second location at two or more time points.

84.-85. (canceled)

86. The method of claim 74, comprising identifying the first location and the second location from a video image on a camera.

87.-88. (canceled)

89. The method of claim 74, comprising utilizing the tissue probe and the one or more optical radiation detectors mounted to an exterior surface in the environment of the mammalian subject.

90.-101. (canceled)

102. A method for remotely determining blood pressure in a mammalian subject comprising:
utilizing one or more tissue probes including one or more pulse detectors responsive to reflected multiple pulses in an environment of the mammalian subject, wherein the reflected multiple pulses to the one or more pulse detectors are operable to measure one or more heart beats of the mammalian subject;
utilizing one or more contact-free tissue probes including one or more optical radiation detectors responsive to reflected optical radiation in an environment of the mammalian subject;
detecting at the one or more optical radiation detectors a first reflected optical radiation from blood at a first location at a skin surface of the subject and a second reflected optical radiation from blood at a second location at the skin surface of the subject;
determining the timing of a first blood pressure pulse at the first location by comparing levels of the first reflected optical radiation measured at different times;
determining the timing of a second blood pressure pulse at the second location by comparing levels of the second reflected optical radiation measured at different times; and
determining the blood pressure in the subject based on a calculation of timing of the one or more heartbeats relative to the timing of the first blood pressure pulse relative to the timing of the second blood pressure pulse.

103. The method of claim 102, wherein an origin of the first blood pressure pulse and the second blood pressure pulse are a single origin.

104. The method of claim 102, comprising directing a first optical light source toward the first location for generation of the first reflected optical radiation.

105. The method of claim 102, comprising directing a second optical light source toward the second location for generation of the second reflected optical radiation.

106. (canceled)

107. The method of claim 102, wherein the first and second reflected optical radiation are two or more wavelengths of infrared radiation.

108. The method of claim 102, wherein the optical radiation comprises ambient room radiation, ambient solar radiation, artificial room radiation, radiation from an optical light source, LED radiation, incandescent radiation, or fluorescent radiation.

109. (canceled)

110. The method of claim 102, wherein the optical light source comprises an incoherent optical source.

111. The method of claim 102, comprising detecting the first and second reflected optical radiation from the first location and the second location at two or more time points.

112.-113. (canceled)

114. The method of claim 102, comprising measuring systole and diastole at the heart of the subject with the reflected multiple pulses to the one or more pulse detectors.

115.-116. (canceled)

117. The method of claim 102, comprising identifying the first location and the second location relative to a location of the heart of the subject, and applying a corrected calculation to determine the blood pressure in the subject.

118. (canceled)

119. The method of claim 102, comprising utilizing the tissue probe and the one or more optical radiation detectors mounted to an exterior surface in the environment of the mammalian subject.

120.-135. (canceled)
